(19) United States
(12) Patent Application Publication
(10) Pub. No.: US 2007/0249592 A1
(43) Pub. Date: Oct. 25, 2007

(54) Use of selected CGRP antagonists in treatment and prevention of hot flushes in prostate cancer patients

(76) Inventors: Klaus Rudolf, Warthausen (DE); Henri Doods, Warthausen (DE); Stephan Georg Mueller, Warthausen (DE); Annette Zamponi, Biberach (DE); Philipp Lustenberger, Warthausen (DE); Kirsten Arndt, Biberach (DE); Gerhard Schaenzle, Biberach-Mettenberg (DE); Dirk Stenkamp, Biberach (DE); Rolf-Stefan Brickl, Warthausen (DE)

Correspondence Address: MICHAEL P. MORRIS;BOEHRINGER INGELHEIM CORPORATION, 900 RIDGEBURY RD, P. O. BOX 368, RIDGEFIELD, CT, 06877-0368, US

(21) Appl. No.: 11/774,995

(22) Filed: Jul. 9, 2007

Related U.S. Application Data
(63) Continuation of application No. 11/301,422, filed on Dec. 13, 2005.

(30) Foreign Application Priority Data
Dec. 29, 2004 (DE) 102004063755

(51) Int. Cl. A61K 31/517; A61K 31/5513

(52) U.S. Cl. 514/221; 514/266.3

(57) ABSTRACT

The invention relates to a method of treatment or prevention of hot flushes in men who underwent castration, e.g. due to androgen ablation treatment in prostate cancer therapy, comprising administration of an effective amount of a selected CGRP antagonist to the patient, and to the use of said active compounds for the manufacture of a pharmaceutical composition intended to be used in this method.

### TECHNICAL FIELD OF THE INVENTION

The present invention relates to a method of treatment or prevention of hot flushes (also called hot flashes) in men who underwent castration, e.g. due to androgen ablation treatment in prostate cancer therapy, comprising administration of an effective amount of a selected CGRP antagonist to a person in need of such treatment. The method according to the invention preferably comprises monotherapy with a single substance, but also includes combined therapy with a number of substances from the specified group of active substances.

In a second aspect, the invention relates to the use of a selected CGRP antagonist for manufacture of a pharmaceutical composition for prevention or treatment of hot flushes in men who underwent castration.

### BACKGROUND OF THE INVENTION

Hot flushes and sweating, that is vasomotor symptoms, are reported by 43 to 77% of prostate cancer patients after medical or surgical castration, usually persisting for many years, possibly impairing quality of life (Arch. Surg. 43: 209, 1941; J. Urol. 152: 1170, 1994). Furthermore, hot flushes occur in 75% of women after menopause. In WO 01/10425 it has been proposed that the symptoms of menopausal hot flushes can be effectively prevented or their distressing effects substantially alleviated by substances which antagonise the effects of CGRP (CGRP antagonists) or inhibit or reduce the release of CGRP from sensory nerve endings (CGRP release inhibitors), this therapeutic approach being superior to hormone replacement therapy in particular because of its lack of side effects.

Although it has been already reported that plasma calcitonin gene-related peptide was increased during hot flushes in six men who underwent castration therapy, the mechanism of hot flushes in men is not well known. For instance, it is unclear up to now why some men have vasomotor symptoms whereas some do not and it was suggested to discover more about the mechanism of these symptoms to develop new treatment alternatives (J. Urol. 166: 1720-1723, 2001).

### BRIEF SUMMARY OF THE INVENTION

There is a clear need for alternative approaches and improvement in the treatment and prevention of hot flushes in men who underwent castration.

It is therefore an object of the invention to provide a method of treatment and prevention of hot flushes in men who underwent castration, comprising administering to a patient in need of such treatment an effective amount of a selected CGRP antagonist.

A second object of the invention is the use of a selected CGRP antagonist for manufacture of a pharmaceutical composition for prevention or treatment of hot flushes in men who underwent castration.

### DETAILED DESCRIPTION OF THE INVENTION

It has now been found that the symptoms of hot flushes in men who underwent castration can be effectively prevented or their distressing effects substantially alleviated by substances which antagonise the effects of CGRP (CGRP antagonists), this therapeutic approach being superior to conventional therapy.

The present invention thus relates to the use of selected CGRP antagonists for combating hot flushes in men who underwent castration, including both prevention and acute treatment. The use according to the invention preferably comprises monotherapy with a single substance, but also includes combined therapy with a number of substances from the specified groups of active substances. Moreover, the treatment according to the invention may be carried out in addition to conventional therapy.

The CGRP antagonists according to the present invention which may be used for the treatment and/or prevention of hot flushes in men who underwent castration, for the preparation of a corresponding pharmaceutical composition, are selected from the group consisting of
(1) 4-(2-oxo-1,2,4,5-tetrahydro-1,3-benzdiazepin-3-yl)-piperidine-1-carboxylic acid {(R)-1-(4-amino-3-chloro-5-ethyl-benzyl)-2-[4-(4-methyl-piperazine-1-yl)-piperidine-1-yl]-2-oxo-ethyl}-amide, 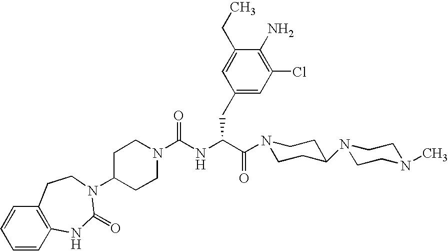
(2) [1′-((R)-3-(4-amino-3-chloro-5-trifluoromethyl-phenyl)-2-{[4-(2-oxo-1,2,4,5-tetrahydro-1,3-benzdiazepin-3-yl)-piperidine-1-carbonyl]-amino}-propionyl)-4,4′-bipiperidinyl-1-yl]-acetic acid, 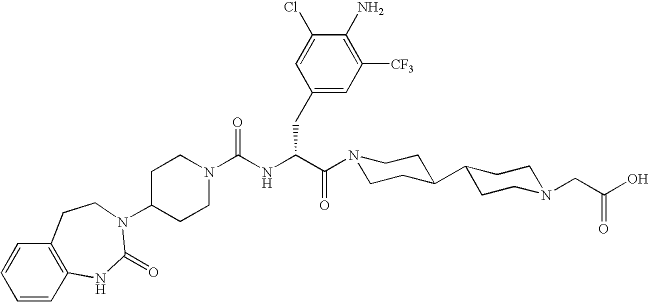
(3) 3-{1-[(R)-1-(4-amino-3,5-dibromo-benzyl)-2-[1,4′]bipiperidinyl-1′-yl-2-oxo-ethylcarbamoyl]-piperidine-4-yl}-2-oxo-1,2,3,4-tetrahydro-chinazolin-7-carboxylic acid, 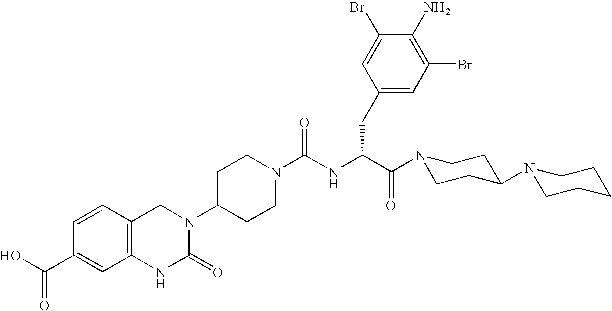
(4) 4-(2-oxo-1,2,4,5-tetrahydro-1,3-benzdiazepin-3-yl)-piperidine-1-carboxylic acid (R)-1-(7-methyl-1H-benztriazol-5-ylmethyl)-2-[4-(4-methyl-piperazine-1-yl)-piperidine-1-yl]-2-oxo-ethyl ester, 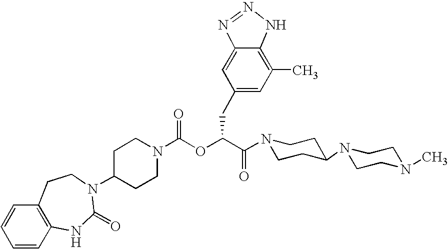
(5) (S)-2-(3-chloro-4-hydroxy-5-trifluoromethyl-benzyl)-1-[4-(1-methyl-piperidine-4-yl)-piperazine-1-yl]-4-[4-(2-oxo-1,2,4,5-tetrahydro-1,3-benzdiazepin-3-yl)-piperidine-1-yl]-butane-1,4-dione, 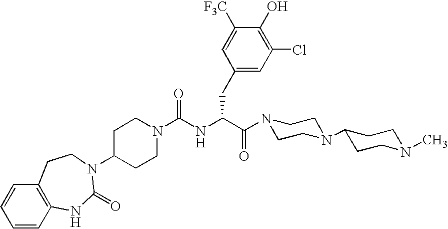
(6) 4-(2-oxo-1,2,4,5-tetrahydro-1,3-benzdiazepin-3-yl)-piperidine-1-carboxylic acid (R)-1-(4-hydroxy-3,5-dimethyl-benzyl)-2-oxo-2-(4-piperidine-4-yl-piperazine-1-yl)-ethyl ester, 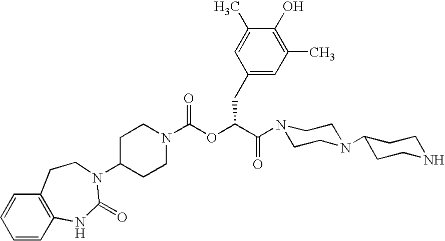
(7) 4-(2-oxo-1,2,4,5-tetrahydro-1,3-benzdiazepin-3-yl)-piperidine-1-carboxylic acid (R)-1-(3,5-dibromo-4-hydroxy-benzyl)-2-[4-(1-methyl-piperidine-4-yl)-piperazine-1-yl]-2-oxo-ethyl ester, 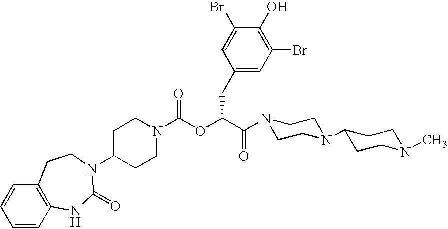
(8) 4-(2-oxo-1,2,4,5-tetrahydro-1,3-benzdiazepin-3-yl)-piperidine-1-carboxylic acid (R)-1-(6-amino-5-methyl-pyridine-3-ylmethyl)-2-oxo-2-(4-piperazine-1-yl-piperidine-1-yl)-ethyl ester, 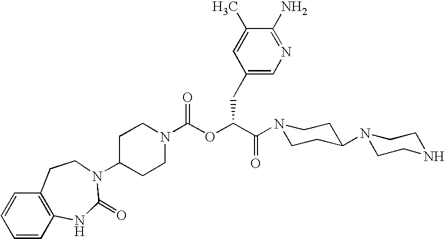
(9) 4-(2-oxo-1,2,4,5-tetrahydro-1,3-benzdiazepin-3-yl)-piperidine-1-carboxylic acid (R)-1-(3,5-dibromo-4-hydroxy-benzyl)-2-oxo-2-(4-piperazine-1-yl-piperidine-1-yl)-ethyl ester, 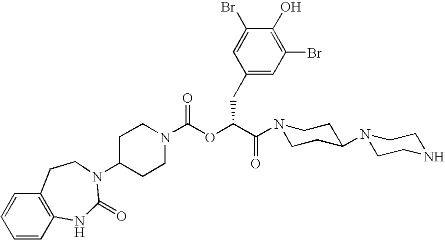
(10) 4-(2-oxo-1,2,4,5-tetrahydro-1,3-benzdiazepin-3-yl)-piperidine-1-carboxylic acid (R)-1-(3,5-dibromo-4-hydroxy-benzyl)-2-oxo-2-(4-piperidine-4-yl-piperazine-1-yl)-ethyl ester, 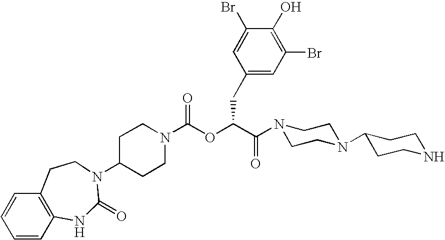
(11) (S)-2-(4-amino-3-chloro-5-trifluoromethyl-benzyl)-1-[4-(4-methyl-piperazine-1-yl)-piperidine-1-yl]-4-[4-(2-oxo-1,2,4,5-tetrahydro-1,3-benzodiazepin-3-yl)-piperidine-1-yl]-butane-1,4-dione, 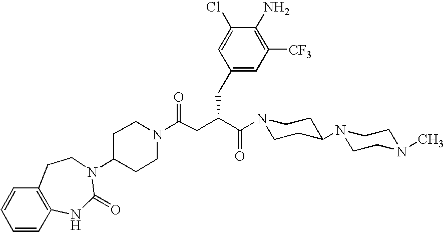
(12) 4-(2-oxo-1,2,4,5-tetrahydro-1,3-benzodiazepin-3-yl)-piperidine-1-carboxylic acid {(R)-1-(3,4-diethyl-benzyl)-2-[4-(1-methyl-piperidine-4-yl)-piperazine-1-yl]-2-oxo-ethyl}-amide, 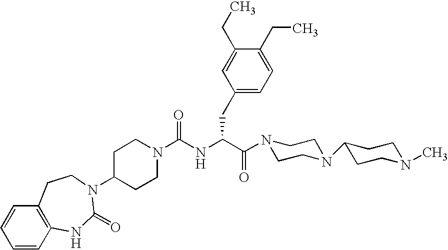
(13) 4-(2-oxo-1,2,4,5-tetrahydro-1,3-benzodiazepin-3-yl)-piperidine-1-carboxylic acid (R)-1-(4-amino-3-chloro-5-trifluoromethyl-benzyl)-2-[4-(4-methyl-piperazine-1-yl)-piperidine-1-yl]-2-oxo-ethyl ester, 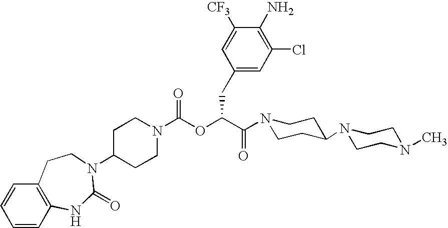
(14) 4-(2-oxo-1,2,4,5-tetrahydro-1,3-benzodiazepin-3-yl)-piperidine-1-carboxylic acid {(R)-1-(4-amino-3-chloro-5-trifluoromethyl-benzyl)-2-[4-(4-methyl-piperazine-1-yl)-piperidine-1-yl]-2-oxo-ethyl}-amide, 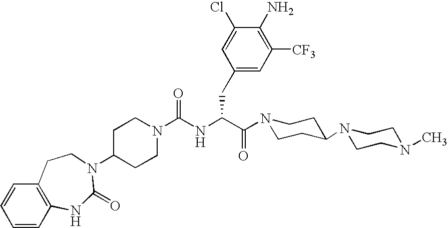
(15) ((S)-2-(4-amino-3,5-bis-trifluoromethyl-benzyl)-1-[4-(4-methyl-piperazine-1-yl)-piperidine-1-yl]-4-[4-(2-oxo-1,2,4,5-tetrahydro-1,3-benzodiazepin-3-yl)-piperidine-1-yl]-butane-1,4-dione, 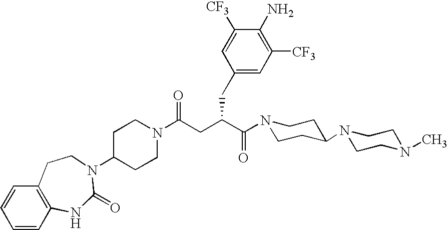
(16) 4-(2-oxo-1,2,4,5-tetrahydro-1,3-benzodiazepin-3-yl)-piperidine-1-carboxylic acid {(R)-1-(4-amino-3,5-bis-trifluoromethyl-benzyl)-2-[4-(4-methyl-piperazine-1-yl)-piperidine-1-yl]-2-oxo-ethyl}-amide, 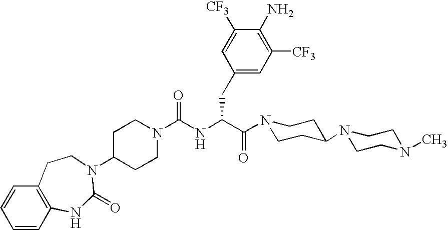
(17) 4-(2-oxo-1,2,4,5-tetrahydro-1,3-benzodiazepin-3-yl)-piperidine-1-carboxylic acid (R)-1-(4-amino-3,5-bis-trifluoromethyl-benzyl)-2-[4-(4-methyl-piperazine-1-yl)-piperidine-1-yl]-2-oxo-ethyl ester, 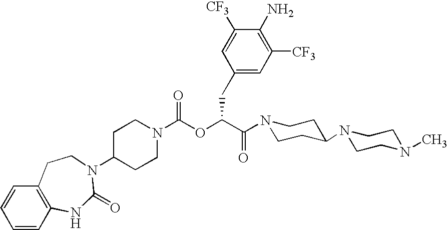
(18) 4-(2-oxo-1,2,4,5-tetrahydro-1,3-benzdiazepin-3-yl)-piperidine-1-carboxylic acid {(R)-1-(4-amino-3-chloro-5-methyl-benzyl)-2-[4-(1-methyl-piperidine-4-yl)-piperazine-1-yl]-2-oxo-ethyl}-amide, 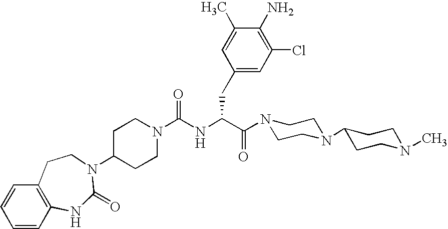
(19) 4-(2-oxo-1,2,4,5-tetrahydro-1,3-benzodiazepin-3-yl)-piperidine-1-carboxylic acid (R)-1-(3,5-dibromo-4-hydroxy-benzyl)-2-[4-(4-methyl-piperazine-1-yl)-piperidine-1-yl]-2-oxo-ethyl ester, 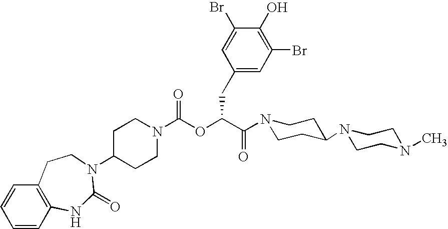
(20) 4-(2-oxo-1,2,4,5-tetrahydro-1,3-benzdiazepin-3-yl)-piperidine-1-carboxylic acid (R)-1-(4-hydroxy-3,5-dimethyl-benzyl)-2-oxo-2-(4-piperazine-1-yl-piperidine-1-yl)-ethyl ester, 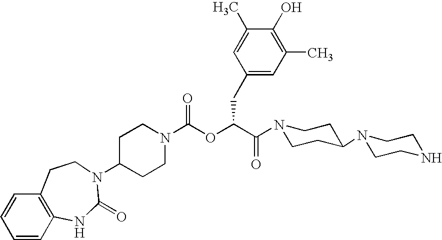
(21) 4-(2-oxo-1,2,4,5-tetrahydro-1,3-benzdiazepin-3-yl)-piperidine-1-carboxylic acid (R)-1-(4-hydroxy-3,5-dimethyl-benzyl)-2-[4-(1-methyl-piperidine-4-yl)-piperazine-1-yl]-2-oxo-ethyl ester, 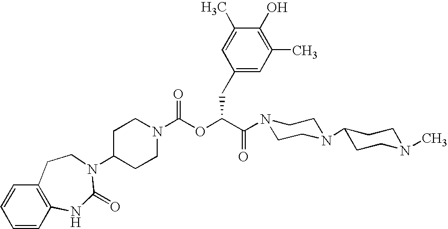
(22) (S)-1-1,4′-bipiperidinyl-1′-yl-2-(3-chloro-4-hydroxy-5-trifluoromethyl-benzyl)-4-[4-(2-oxo-1,2,4,5-tetrahydro-1,3-benzdiazepin-3-yl)-piperidine-1-yl]-butane-1,4-dione, 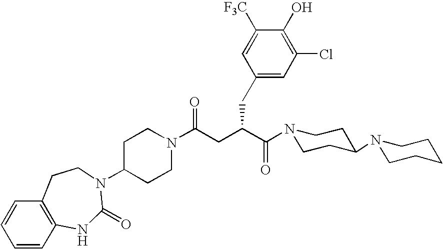
   the physiologically acceptable salts thereof and the hydrates of the salts.

The dosage required to produce the desired effect is appropriately 0.0001 to 3 mg/kg of body weight, preferably 0.01 to 1 mg/kg of body weight, for intravenous or subcutaneous administration, 0.01 to 20 mg/kg of body weight, preferably 0.1 to 20 mg/kg of body weight, for oral administration and 0.01 to 10 mg/kg of body weight, preferably 0.1 to 10 mg/kg of body weight, by nasal route or by inhalation, 1 to 3 times a day in each case.

If the treatment with the selected CGRP antagonists is given as a supplement to conventional therapy, it is advisable to reduce the doses given above, and in this case the dosage may range from 1/5 of the lower limits specified above up to 1/1 of the upper limits specified above.

For this purpose, the selected CGRP antagonists, the physiologically acceptable salts thereof or the hydrates of said salts may be formulated with one or more conventional inert carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethyleneglycol, propylene glycol, cetylstearyl alcohol, carboxymethylcellulose or fatty substances such as hard fat or suitable mixtures thereof in conventional galenic preparations such as plain or coated tablets, capsules, powders, suspensions, solutions, metering aerosols or suppositories.

Preparations which are particularly suitable for the method of treatment or prevention according to the invention are those which contain one of the selected CGRP antagonists, a physiologically acceptable salt thereof or a hydrate of said salt.

in one of the following pharmaceutical formulations:

capsules for powder inhalation containing 1 mg of active substance,

inhalable solution for nebulisers containing 1 mg of active substance,

propellant gas-operated metering aerosol containing 1 mg of active substance,

nasal spray containing 1 mg of active substance,

tablets containing 20 mg of active substance,

capsules containing 20 mg of active substance,

aqueous solution for nasal application containing 10 mg of active substance,

aqueous solution for nasal application containing 5 mg of active substance, or

suspension for nasal application containing 20 mg of active substance.

In the method according to the invention and in any of the formulations given the selected CGRP antagonist may also be used in form of a physiologically acceptable salt or a hydrate of said salt. Amounts are given based on the free base.

CGRP is released by sensory nerves, e.g. the trigeminal nerve which innervates part of the skin of the face. It has already been shown that stimulation of the trigeminal ganglion in humans leads to an increase in the CGRP plasma level and causes reddening of the face ([4]: P. J. Goadsby et al., Annals of Neurology, Vol. 23, No. 2, 1988, 193-196,).

To demonstrate that hot flushes can be successfully treated using CGRP antagonists, an increased release of endogenous CGRP was induced in marmosets by stimulating the trigeminal ganglion, leading to increased blood flow through the blood vessels of the skin. The efficacy of the following test substances was characterised by determining the dose administered i.v. which reduces by 50% the increased blood flow through the skin of the face which has been brought about by endogenous CGRP:
(1) (S)-2-(4-amino-3-chloro-5-trifluoromethyl-benzyl)-1-[4-(4-methyl-piperazin-1-yl)-piperidin-1-yl]-4-[4-(2-oxo-1,2,4,5-tetrahydro-1,3-benzodiazepin-3-yl)-piperidin-1-yl]-butane-1,4-dione,
(2) 4-(2-oxo-1,2,4,5-tetrahydro-1,3-benzodiazepin-3-yl)-piperidine-1-carboxylic acid {(R)-1-(3,4-diethyl-benzyl)-2-[4-(1-methyl-piperidin-4-yl)-piperazin-1-yl]-2-oxo-ethyl}-amide,
(3) 4-(2-oxo-1,2,4,5-tetrahydro-1,3-benzodiazepin-3-yl)-piperidine-1-carboxylic acid (R)-1-(4-amino-3-chloro-5-trifluoromethyl-benzyl)-2-[4-(4-methyl-piperazin-1-yl)-piperidin-1-yl]-2-oxo-ethyl ester,
(4) 4-(2-oxo-1,2,4,5-tetrahydro-1,3-benzodiazepin-3-yl)-piperidine-1-carboxylic acid {(R)-1-(4-amino-3-chloro-5-trifluoromethyl-benzyl)-2-[4-(4-methyl-piperazin-1-yl)-piperid-1-yl]-2-oxo-ethyl}-amide,
(5) (S)-2-(4-amino-3,5-bis-trifluoromethyl-benzyl)-1-[4-(4-methyl-piperazin-1-yl)-piperidin-1-yl]-4-[4-(2-oxo-1,2,4,5-tetrahydro-1,3-benzodiazepin-3-yl)-piperidin-1-yl]-butane-1,4-dione,
(6) 4-(2-oxo-1,2,4,5-tetrahydro-1,3-benzodiazepin-3-yl)-piperidine-1-carboxylic acid {(R)-1-(4-amino-3,5-bis-trifluoromethyl-benzyl)-2-[4-(4-methyl-piperazin-1-yl)-piperidin-1-yl]-2-oxo-ethyl}-amide,
(7) 4-(2-oxo-1,2,4,5-tetrahydro-1,3-benzodiazepin-3-yl)-piperidine-1-carboxylic acid (R)-1-(4-amino-3,5-bis-trifluoromethyl-benzyl)-2-[4-(4-methyl-piperazin-1-yl)-piperidin-1-yl]-2-oxo-ethyl ester,
(8) sumatriptan and
(9) zolmitriptan.
   Description of Method:

Marmosets of both sexes (300-400 g) are anaesthetised with pentobarbital (initially with 30 mg/kg, i.p., followed by infusion of 6 mg/kg/h, i.m.). The body temperature is maintained at 37° C. using a heating die base. Pancurmium is administered as a muscle relaxant (initially 1 mg/kg, 0.5 mg after each hour thereafter). The animal's head is secured in a stereotactical apparatus. After the skin on the head has been opened using a lengthwise incision, a small hole is drilled in the skull and a bipolar electrode (Rhodes SNES 100) is lowered into the trigeminal ganglion.

Locating the ganglion is made easier by the use of an X-ray which shows up the bone structure of the skull. The petrous bone serves as a guide for placing the electrode (CCX-Digital X-ray apparatus). The position of the electrode in the ganglion is monitored at the end of each experiment. The stimulation parameters are:

10 Hz, 2 mA, 2 msec, for 30 sec.

The blood flow in the micro-vessels of the facial skin is determined by laser Doppler flow measurement using a PeriFlux Laser Doppler System.

The animals are exposed to 2 to 3 stimulation periods at intervals of 30 minutes in each case. The first stimulation serves as a reference value for the other stimulations. The test substances are administered i.v. 5 minutes before the 2nd and 3rd stimulation periods.

The Examples which follow describe pharmaceutical preparations which contain as active substance a selected CGRP antagonist according to the present invention for use according to the invention, a physiologically acceptable salt thereof or a hydrate of the salt. In the following table, the mentioned CGRP antagonists are numbered for identification of active ingredients in the tables of the examples.

Active Ingredients

| Subst. No. | Substance |
|---|---|
| 1 | CGRP-Antagonist (1) or a physiologically acceptable salt thereof or a |
| | hydrate of said salt [1a] |
| 2 | CGRP-Antagonist (2) a physiologically acceptable salt thereof or a hydrate |
| | of said salt [2a] |
| 3 | CGRP-Antagonist (3) a physiologically acceptable salt thereof or a hydrate |
| | of said salt [3a] |
| 4 | CGRP-Antagonist (4) a physiologically acceptable salt thereof or a hydrate |
| | of said salt [4a] |
| 5 | CGRP-Antagonist (5) a physiologically acceptable salt thereof or a hydrate |
| | of said salt [5a] |
| 6 | CGRP-Antagonist (6) a physiologically acceptable salt thereof or a hydrate |
| | of said salt [6a] |
| 7 | CGRP-Antagonist (7) a physiologically acceptable salt thereof or a hydrate |
| | of said salt [7a] |
| 8 | CGRP-Antagonist (8) a physiologically acceptable salt thereof or a hydrate |
| | of said salt [8a] |
| 9 | CGRP-Antagonist (9) a physiologically acceptable salt thereof or a hydrate |
| | of said salt [9a] |
| 10 | CGRP-Antagonist (10) a physiologically acceptable salt thereof or a hydrate |
| | of said salt [10a] |
| 11 | CGRP-Antagonist (11) a physiologically acceptable salt thereof or a hydrate |
| | of said salt [11a] |
| 12 | CGRP-Antagonist (12) a physiologically acceptable salt thereof or a hydrate |
| | of said salt [12a] |
| 13 | CGRP-Antagonist (13) a physiologically acceptable salt thereof or a hydrate |
| | of said salt [13a] |
| 14 | CGRP-Antagonist (14) a physiologically acceptable salt thereof or a hydrate |
| | of said salt [14a] |
| 15 | CGRP-Antagonist (15) a physiologically acceptable salt thereof or a hydrate |
| | of said salt [15a] |
| 16 | CGRP-Antagonist (16) a physiologically acceptable salt thereof or a hydrate |
| | of said salt [16a] |
| 17 | CGRP-Antagonist (17) a physiologically acceptable salt thereof or a hydrate |
| | of said salt [17a] |
| 18 | CGRP-Antagonist (18) a physiologically acceptable salt thereof or a hydrate |
| | of said salt [18a] |
| 19 | CGRP-Antagonist (19) a physiologically acceptable salt thereof or a hydrate |
| | of said salt [19a] |
| 20 | CGRP-Antagonist (20) a physiologically acceptable salt thereof or a hydrate |
| | of said salt [20a] |
| 21 | CGRP-Antagonist (21) a physiologically acceptable salt thereof or a hydrate |
| | of said salt [21a] |
| 22 | CGRP-Antagonist (22) a physiologically acceptable salt thereof or a hydrate |
| | of said salt [22a] |

### EXAMPLE 1a

Tablets for 100 mg CGRP-antagonist
Composition/tablet:

| | | |
|---|---|---|
| CGRP-antagonist | 100 | mg |
| Lactose | 375 | mg |
| Magnesiumstearate | 3.0 | mg |
| Povidone | 8.5 | mg |
| Crospovidone | 14.4 | mg |
| Volatile component: water | | |

Method of Preparation:

CGRP-antagonist and Lactose (fine) are mixed homogenously in an adequate mixer (e.g. Diosna P2); afterwards the compound is granulated with an aqueous solution of Povidone. The granulate material is screened with a Kressner sieve (1.6 mm) and dried for 2 h at 40° C. After that, the granulate material is sieved at 3000 U/minute with a mesh size of 1.1 mm in an adequate mill (e.g. Comill). Thereupon the granulate material is first mixed with Crospovidone for five minutes and afterwards with magnesiumstearate for one minute. The finished composition are pressed in a tablet press with an adequate diameter.

### EXAMPLE 1b

Tablets for 10 mg CGRP-antagonist
Composition/tablet:

| | | |
|---|---|---|
| CGRP-antagonist | 10.0 | mg |
| Lactose | 475 | mg |
| Magnesiumstearate | 3.0 | mg |
| Povidone | 8.5 | mg |
| Crospovidone | 14.4 | mg |
| Volatile component: water | | |

Method of Preparation:

CGRP-antagonist and Lactose (fine) are mixed homogenously in an adequate mixer (e.g. Diosna P2); afterwards the compound is granulated with an aqueous solution of Povidone. The granulate material is screened with a Kressner sieve (1.6 mm) and dried for 2 h at 40° C. After that, the granulate material is sieved at 3000 U/minute with a mesh size of 1.1 mm in an adequate mill (e.g. Comill). Thereupon the granulate material is first mixed with Crospovidone for five minutes and afterwards with magnesiumstearate for one minute. The finished composition are pressed in a tablet press with an adequate diameter.

### EXAMPLE 1c

Tablets for 600 mg CGRP-antagonist
Composition/Tablet:

| | | |
|---|---|---|
| CGRP-antagonist | 600 | mg |
| Lactose | 175 | mg |
| Magnesiumstearate | 6 | mg |
| Povidone | 17 | mg |
| Crospovidone | 28.8 | mg |
| Volatile component: water | | |

Method of Preparation:

CGRP-antagonist and Lactose (fine) are mixed homogenously in an adequate mixer (e.g. Diosna P2); afterwards the compound is granulated with an aqueous solution of Povidone; The granulate material is screened with a Kressner sieve (1.6 mm) and dried for 2 h at 40° C. After that, the granulate material is sieved at 3000 U/minute with a mesh size of 1.1 mm in an adequate mill (e.g. Comill). Thereupon the granulate material is first mixed with Crospovidone for five minutes and afterwards with magnesiumstearate for one minute. The finished composition are pressed in a tablet press with an adequate diameter.

### EXAMPLE 1d

Tablets for 100 mg CGRP-antagonist
Composition/Tablet:

| | | |
|---|---|---|
| CGRP-antagonist | 100 | mg |
| Lactose | 403 | mg |
| Magnesiumstearate | 3.1 | mg |
| Povidone | 9.1 | mg |
| Crospovidone | 15.3 | mg |
| Volatile component: water | | |

Method of Preparation:

CGRP-antagonist and Lactose (fine) are mixed homogenously in an adequate mixer (e.g. Diosna P2); afterwards the compound is granulated with an aqueous solution of Povidone. The granulate material is screened with a Kressner sieve (1.6 mm) and dried for 2 h at 40° C. After that, the granulate material is sieved at 3000 U/minute with a mesh size of 1.1 mm in an adequate mill (e.g. Comill). Thereupon the granulate material is first mixed with Crospovidone for five minutes and afterwards with magnesiumstearate for one minute. The finished composition is pressed in a tablet press with an adequate diameter.

The described methods of preparation are the basic principle of further examples shown in the following table.

In the examples 10-600 mg CGRP-antagonist as active form, as a physiologically acceptable salt thereof or a hydrate of said salt is used.

Table of Example 1a-d

| | | | | | | mg | |
|---|---|---|---|---|---|---|---|
| | Substance | | mg | mg | mg | Magnesium- | |
| Expl. | No. | mg | Lactose | Povidone | Crospovidone | stearate | Ø [mm] |
| 1.1 | 3 | 40 | 80.0 | 1.8 | 3.0 | 0.6 | 7 |
| 1.2 | 6a | 100 | 200.0 | 4.5 | 7.6 | 1.6 | 9 |
| 1.3 | 11a | 70 | 140.0 | 3.2 | 5.3 | 1.1 | 8 |
| 1.4 | 2 | 180 | 360.0 | 8.1 | 13.7 | 2.8 | 12 |
| 1.5 | 6 | 120 | 240.0 | 5.4 | 9.1 | 1.9 | 10 |
| 1.6 | 3 | 10 | 70.0 | 1.2 | 2.0 | 0.4 | 6 |
| 1.7 | 17 | 270 | 540.0 | 12.2 | 20.6 | 4.2 | 13 |
| 1.8 | 3a | 220 | 440.0 | 9.9 | 16.7 | 3.4 | 13 |
| 1.9 | 14 | 140 | 280.0 | 6.3 | 10.7 | 2.2 | 11 |
| 1.10 | 5 | 230 | 460.0 | 10.4 | 17.5 | 3.6 | 13 |
| 1.11 | 1 | 230 | 460.0 | 10.4 | 17.5 | 3.6 | 13 |
| 1.12 | 16 | 40 | 80.0 | 1.8 | 3.0 | 0.6 | 7 |
| 1.13 | 3 | 80 | 160.0 | 3.6 | 6.1 | 1.2 | 9 |
| 1.14 | 4a | 320 | 540.0 | 12.9 | 21.8 | 4.5 | 13 |
| 1.15 | 13 | 340 | 580.0 | 13.8 | 23.3 | 4.8 | 13 |
| 1.16 | 21 | 170 | 340.0 | 7.7 | 12.9 | 2.7 | 12 |
| 1.17 | 1a | 110 | 220.0 | 5.0 | 8.4 | 1.7 | 11 |
| 1.18 | 5 | 170 | 340.0 | 7.7 | 12.9 | 2.7 | 12 |
| 1.19 | 5a | 320 | 540.0 | 12.9 | 21.8 | 4.5 | 13 |
| 1.20 | 4 | 30 | 60.0 | 1.4 | 2.3 | 0.5 | 6 |
| 1.21 | 2a | 600 | 600.0 | 18.0 | 30.5 | 6.2 | 13 |
| 1.22 | 15 | 300 | 600.0 | 13.5 | 22.8 | 4.7 | 13 |
| 1.23 | 7 | 160 | 320.0 | 7.2 | 12.2 | 2.5 | 12 |
| 1.24 | 7a | 160 | 320.0 | 7.2 | 12.2 | 2.5 | 12 |
| 1.25 | 4 | 170 | 340.0 | 7.7 | 12.9 | 2.7 | 12 |

### EXAMPLE 2a

Tablets for 100 mg CGRP-antagonist
Composition:

| | | |
|---|---|---|
| CGRP-antagonist | 100 | mg |
| Lactose | 284 | mg |
| Microcrystalline cellulose | 95 | mg |
| Magnesiumstearate | 7.2 | mg |
| Croscarmellose | 7.3 | mg |
| Volatile component: water | | |

Method of Preparation:

CGRP-antagonist, Lactose (fine) and microcrystalline cellulose are mixed homogenously in an adequate mixer (e.g. Diosna P2); afterwards the compound is granulated with water. The granulate material is screened with a Kressner sieve (1.6 mm) and dried for 2 h at 40° C. After that, the granulate material is sieved at 3000 U/minute with a mesh size of 1.1 mm in an adequate mill (e.g. Comill). Thereupon the granulate material is first mixed with Croscarmellose for five minutes and afterwards with magnesiumstearate for one minute. The finished composition are pressed in a tablet press with an adequate diameter.

### EXAMPLE 2b

Tablets for 10 mg CGRP-antagonist
Composition:

| | | |
|---|---|---|
| CGRP-antagonist | 10.0 | mg |
| Lactose | 274 | mg |
| Microcrystalline Cellulose | 109.5 | mg |
| Magnesiumstearate | 7.2 | mg |
| Croscarmellose | 7.3 | mg |
| Volatile component: water | | |

Method of Preparation:

CGRP-antagonist, Lactose (fine) and microcrystalline cellulose are mixed homogenously in an adequate mixer (e.g. Diosna P2); afterwards the compound is granulated with Water. The granulate material is screened with a Kressner sieve (1.6 mm) and dried for 2 h at 40° C. After that, the granulate material is sieved at 3000 U/minute with a mesh size of 1.1 mm in an adequate mill (e.g. Comill). Thereupon the granulate material is first mixed with Croscarmellose for five minutes and afterwards with magnesiumstearate for one minute. The finished composition are pressed in a tablet press with an adequate diameter.

### EXAMPLE 2c

Tablets for 400 mg CGRP-antagonist
Composition:

| | | |
|---|---|---|
| CGRP-antagonist | 400 | mg |
| Lactose | 194 | mg |
| Microcrystalline cellulose | 95 | mg |
| Magnesiumstearate | 7.2 | mg |
| Croscarmellose | 7.3 | mg |
| Volatile component: water | | |

Method of Preparation:

CGRP-antagonist, Lactose (fine) and microcrystalline cellulose are mixed homogenously in an adequate mixer (e.g. Diosna P2); afterwards the compound is granulated with Water. The granulate material is screened with a Kressner sieve (1.6 mm) and dried for 2 h at 40° C. After that, the granulate material is sieved at 3000 U/minute with a mesh size of 1.1 mm in an adequate mill (e.g. Comill). Thereupon the granulate material is first mixed with Croscarmellose for five minutes and afterwards with magnesiumstearate for one minute. The finished composition are pressed in a tablet press with an adequate diameter.

### EXAMPLE 2d

Tablets for 100 mg CGRP-antagonist
Composition:

| | | |
|---|---|---|
| CGRP-antagonist | 100 | mg |
| Lactose | 403 | mg |
| Microcrystalline Cellulose | 12.1 | mg |
| Magnesiumstearate | 9.3 | mg |
| Croscarmellose | 9.4 | mg |
| Volatile component: water | | |

Method of Preparation:

CGRP-antagonist, Lactose (fine) and microcrystalline cellulose are mixed homogenously in an adequate mixer (e.g. Diosna P2); afterwards the compound is granulated with Water. The granulate material is screened with a Kressner sieve (1.6 mm) and dried for 2 h at 40° C. After that, the granulate material is sieved at 3000 U/minute with a mesh size of 1.1 mm in an adequate mill (e.g. Comill). Thereupon the granulate material is first mixed with Croscarmellose for five minutes and afterwards with magnesiumstearate for one minute. The finished composition are pressed in a tablet press with an adequate diameter.

These methods of preparation are the basic principle of further examples being shown in the following table.

In the examples 10-600 mg CGRP-antagonist as active form, as a physiologically acceptable salt thereof or a hydrate of said salt is used.

Table of Example 2a-d

| | | | | mg | | | |
|---|---|---|---|---|---|---|---|
| | Substanz | | mg | mikrokrist. | mg Mg- | mg Cros- | |
| Example | No. | mg | Lactose | Cellulose | stearate | carmellose | Ø [mm] |
| 2.1 | 5 | 130 | 195.0 | | 5.0 | 5.0 | 10 |
| 2.2 | 4a | 380 | 570.0 | 190.0 | 14.5 | 14.8 | 13 |
| 2.3 | 16 | 150 | 225.0 | 75.0 | 5.7 | 5.8 | 10 |
| 2.4 | 6a | 240 | 360.0 | 120.0 | 9.2 | 9.3 | 12 |
| 2.5 | 6 | 30 | 45.0 | 15.0 | 1.1 | 1.2 | 6 |
| 2.6 | 3 | 600 | 400.0 | 200.0 | 15.3 | 15.5 | 13 |
| 2.7 | 2a | 220 | 330.0 | 110.0 | 8.4 | 8.5 | 12 |
| 2.8 | 22 | 30 | 45.0 | 15.0 | 1.1 | 1.2 | 6 |
| 2.9 | 4 | 120 | 180.0 | 60.0 | 4.6 | 4.7 | 9 |
| 2.10 | 2 | 40 | 60.0 | 20.0 | 1.5 | 1.6 | 6 |
| 2.11 | 1 | 110 | 165.0 | 55.0 | 4.2 | 4.3 | 9 |
| 2.12 | 5a | 180 | 270.0 | 90.0 | 6.9 | 7.0 | 12 |
| 2.13 | 6 | 310 | 465.0 | 155.0 | 11.9 | 12.0 | 13 |
| 2.14 | 1 | 390 | 585.0 | 195.0 | 14.9 | 15.1 | 13 |
| 2.15 | 1a | 10 | 150.0 | 32.0 | 2.4 | 2.5 | 8 |
| 2.16 | 15 | 240 | 360.0 | 120.0 | 9.2 | 9.3 | 13 |
| 2.17 | 7 | 50 | 75.0 | 25.0 | 1.9 | 1.9 | 7 |
| 2.18 | 3 | 90 | 135.0 | 45.0 | 3.4 | 3.5 | 8 |
| 2.19 | 17a | 190 | 285.0 | 95.0 | 7.3 | 7.4 | 12 |
| 2.20 | 6 | 360 | 540.0 | 180.0 | 13.8 | 14.0 | 13 |

### EXAMPLE 3a

Aqueous solution for nasal administration of 20% CGRP-antagonist
Composition:

| | | |
|---|---|---|
| CGRP-antagonist | 20 | mg |
| Mannitol | 5 | mg |
| Water | ad 0.1 | ml |

Method of Preparation:

The active ingredient are dissolved/suspended by stirring and if necessary by heating. After mannitol is added the solution is filled up to the final volume.

### EXAMPLE 3b

Aqueous solution for nasal administration of 2% CGRP-antagonist
Composition:

| | | |
|---|---|---|
| CGRP-antagonist | 2 | mg |
| Mannitol | 5 | mg |
| Water | ad 0.1 | ml |

Method of Preparation:

The active ingredient is dissolved/suspended by stirring and if necessary by heating. After mannitol is added the solution is filled up to the final volume.

### EXAMPLE 3c

Aqueous solution for nasal administration of 40% CGRP-antagonist
Composition:

| | | |
|---|---|---|
| CGRP-antagonist | 40 | mg |
| Mannitol | 5 | mg |
| Water | ad 0.1 | ml |

Method of Preparation:

The active ingredient is dissolved/suspended by stirring and if necessary by heating. After mannitol is added the solution is filled up to the final volume.

### EXAMPLE 3d

Aqueous solution for nasal administration of 20%
CGRP-antagonist and 1.5% Labrasol
Composition:

| | | |
|---|---|---|
| CGRP-antagonist | 20 | mg |
| Labrasol | 1.5 | mg |
| Mannitol | 5 | mg |
| Water | ad 0.1 | ml |

Method of Preparation:

The active ingredient is dissolved/suspended by stirring and if necessary by heating. After mannitol and labrasol are added the solution is filled up to the final volume.

### EXAMPLE 3e

Aqueous solution for nasal administration of 50%
CGRP-antagonist and 1.5% Labrasol
Composition:

| | | |
|---|---|---|
| CGRP-antagonist | 50 | mg |
| Rizatriptan | 2 | mg |
| Labrasol | 1.5 | mg |
| Mannitol | 5 | mg |
| Wasser | ad 0.1 | ml |

Method of Preparation:

The active ingredient is dissolved/suspended by stirring and if necessary by heating. After mannitol and labrasol are added the solution is filled up to the final volume.

This method of preparation is the basic principle of further examples being shown in the following table.

Table of Example 3a-e

| | CGRP-Antagonist | | | |
|---|---|---|---|---|
| Example | No. | mg | mg Mannitol | mg Labrasol |
| 3.1 | 3 | 20 | 5 | 3.00 |
| 3.2 | 2 | 10 | 5 | 1.50 |
| 3.3 | 11a | 10 | 5 | 3.00 |
| 3.4 | 5a | 20 | 5 | 1.50 |
| 3.5 | 6 | 10 | 5 | 0.00 |
| 3.6 | 13 | 5 | 5 | 1.50 |
| 3.7 | 4a | 10 | 5 | 3.00 |
| 3.8 | 3a | 5 | 5 | 3.00 |
| 3.9 | 3 | 20 | 5 | 3.00 |
| 3.10 | 1 | 5 | 5 | 0.00 |
| 3.11 | 4 | 10 | 5 | 1.50 |
| 3.12 | 12 | 10 | 5 | 3.00 |
| 3.13 | 4 | 20 | 5 | 3.00 |
| 3.14 | 22 | 5 | 5 | 0.00 |
| 3.15 | 14a | 20 | 5 | 0.00 |

Pellets

Pharmaceutical preparations of CGRP antagonist according to the present invention in form of small particles e.g pellets are also possible. At this the active ingredient is sprayed on neutral starter cores made of saccharose and starch or made of microcrystalline cellulose.

In the case of pH dependent solubility of the active ingredient, alkaline starter cores are used.

The method of preparation includes following steps:

1. Choice/method of preparation of starter cores

2. Spraying of the active ingredient layer

For improvement of stability or flavour or for sustained release the last facultative step is coating of the pellets

### EXAMPLE 4a

Method of preparation of alkaline starter cores:
Composition:

| | |
|---|---|
| Povidone K25 | 3 weight parts |
| Microcrystalline cellulose | 20 weight parts |
| Meglumine | 77 weight parts |

77 Weight parts meglumine, 20 weight parts microcrystalline cellulose and 3 weight parts Povidone K25 are mixed in an adequate mixer for 15 minutes. Afterwards the composition is extruded through a twin screw extruder at a rate of 1 kg/h by metered addition of water. The moment of torsion of 19% is controlled by the proportioning of the water. The diameter of the holes of the die base at the end of the extruder is 0.8 mm.

The spheronizing of the product is made by a spheronizer, for 3 minutes at approx. 850 RPM.

Drying of the pellets at 80° C. for 1.5 h in a fluid bed dryer.

The material is screened by a tumble screener with different sieve die bases (0.71-1.25 mm). The adequate fractions between 0.71 and 0.90 resp. 0.90 and 1.12 mm are used in the following processes.

### EXAMPLE 4b

Method of spraying of 100 mg CGRP-antagonist
Composition:

| | |
|---|---|
| Starter cores | 200 weight parts |
| Hydroxypropylcellulose | 38 weight parts |
| Talcum | 20 weight parts |
| CGRP-antagonist | 100 weight parts |

Hydroxypropylcellulose is solved by stirring in 250 weight parts of 2-propanol. Subsequently the active ingredient and talcum are dispersed in this solution by stirring. 200 weight parts of starter cores are sprayed with the above described dispersion in a fluid bed dryer at an incoming air temperature of 20° C. to 30° C. The pellets are afterwards dried in a drying chamber with circulating air for 8 h at 35° C.

To remove of agglomerated pellets the pellets are sieved through a sieve with a mesh number of 1.25 mm.

### EXAMPLE 4c

Method of spraying of 10 weight parts CGRP-antagonist
Composition:

| | |
|---|---|
| Starter cores | 100 weight parts |
| Hydroxypropylcellulose | 24 weight parts |
| Talcum | 12 weight parts |
| CGRP-antagonist | 10 weight parts |

Hydroxypropylcellulose is solved by stirring in 250 weight parts of 2-propanol. Subsequently the active ingredient and talcum are dispersed in this solution by stirring. 100 weight parts of starter cores are sprayed with the above described dispersion in a fluid bed dryer at an incoming air temperature of 20° C. to 30° C. The pellets are afterwards dried in a drying chamber with circulating air for 8 h at 35° C.

To remove of agglomerated pellets the pellets are sieved through a sieve with a mesh size of 1.25 mm.

### EXAMPLE 4d

Method of spraying of 400 weight parts CGRP-antagonist
Composition:

| | |
|---|---|
| Starter cores | 100 weight parts |
| Hydroxypropylcellulose | 62 weight parts |
| Talcum | 24 weight parts |
| CGRP-antagonist | 400 weight parts |

Hydroxypropylcellulose is solved by stirring in 250 weight parts of 2-propanol. Subsequently the active ingredient and talcum are dispersed in this solution by stirring. 100 weight parts of starter cores are sprayed with the above described dispersion in a fluid bed dryer at an incoming air temperature of 20° C. to 30° C. The pellets are afterwards dried in a drying chamber with circulating air for 8 h at 35° C.

To remove of agglomerated pellets the pellets are sieved through a sieve with a mesh size of 1.25 mm.

In general the build up of the layer of active ingredient is always the same, but variation of the kind and the amount of active ingredient and the excipients is possible.

The following table shows different compositions of the above described method. In the examples 10-600 weight parts CGRP-antagonist as active form, as a physiologically acceptable salt thereof or a hydrate of said salt is used.

Table of Example 4b-d

| | CGRP- | | | | | | *WP. | | |
|---|---|---|---|---|---|---|---|---|---|
| | Antagonist | | *WP | *WP. | *WP. | *WP. | Iso- | *WP. | *WP. |
| Ex. | No. | Wp | Povidone | HPC | Starterpellets | Talkum | propanol | Ethanol | Water |
| 4.1 | 2 | 70 | 14.0 | 0.0 | 70.0 | 15.4 | 2630 | 0 | 0 |
| 4.2 | 22 | 240 | 48.0 | 0.0 | 240.0 | 52.8 | 1600 | 0 | 1600 |
| 4.3 | 5a | 60 | 0.0 | 12.0 | 60.0 | 13.2 | 0 | 1600 | 0 |
| 4.4 | 1a | 230 | 0.0 | 46.0 | 230.0 | 50.6 | 0 | 0 | 1770 |
| 4.5 | 11 | 40 | 0.0 | 8.0 | 450.0 | 49.8 | 4210 | 0 | 0 |
| 4.6 | 12 | 220 | 0.0 | 44.0 | 220.0 | 48.4 | 0 | 0 | 2940 |
| 4.7 | 4 | 380 | 76.0 | 0.0 | 380.0 | 83.6 | 3610 | 0 | 0 |
| 4.8 | 7 | 380 | 0.0 | 76.0 | 380.0 | 83.6 | 2230 | 0 | 0 |
| 4.9 | 4 | 230 | 0.0 | 46.0 | 230.0 | 50.6 | 0 | 1640 | 0 |
| 4.10 | 11a | 360 | 72.0 | 0.0 | 360.0 | 79.2 | 1700 | 0 | 0 |
| 4.11 | 6 | 250 | 0.0 | 50.0 | 250.0 | 55.0 | 0 | 0 | 1760 |
| 4.12 | 4 | 280 | 0.0 | 56.0 | 280.0 | 61.6 | 0 | 1800 | 0 |
| 4.13 | 13 | 360 | 72.0 | 0.0 | 360.0 | 79.2 | 0 | 2400 | 0 |
| 4.14 | 4 | 120 | 0.0 | 24.0 | 360.0 | 50.4 | 0 | 0 | 4950 |
| 4.15 | 14a | 310 | 0.0 | 62.0 | 310.0 | 68.2 | 0 | 0 | 2670 |
| 4.16 | 6 | 600 | 0.0 | 120.0 | 600.0 | 132.0 | 1900 | 0 | 0 |
| 4.17 | 2 | 280 | 56.0 | 0.0 | 280.0 | 61.6 | 2230 | 0 | 0 |
| 4.18 | 17 | 350 | 70.0 | 0.0 | 350.0 | 77.0 | 0 | 1610 | 0 |
| 4.19 | 5 | 10 | 2.0 | 0.0 | 100.0 | 11.2 | 0 | 0 | 1930 |
| 4.20 | 3 | 180 | 0.0 | 36.0 | 180.0 | 39.6 | 1870 | 0 | 0 |
| 4.21 | 14 | 100 | 20.0 | 0.0 | 100.0 | 22.0 | 0 | 1680 | 0 |
| 4.22 | 16a | 80 | 16.0 | 0.0 | 80.0 | 17.6 | 1900 | 0 | 0 |
| 4.23 | 4 | 20 | 0.0 | 4.0 | 350.0 | 37.4 | 0 | 0 | 1930 |
| 4.24 | 6a | 300 | 0.0 | 60.0 | 300.0 | 66.0 | 0 | 2890 | 0 |
| 4.25 | 2 | 290 | 0.0 | 58.0 | 290.0 | 63.8 | 2670 | 0 | 0 |
| 4.26 | 22 | 280 | 56.0 | 0.0 | 280.0 | 61.6 | 1890 | 0 | 0 |
| 4.27 | 3a | 70 | 14.0 | 0.0 | 70.0 | 15.4 | 0 | 3210 | 0 |
| 4.28 | 4a | 50 | 0.0 | 10.0 | 50.0 | 11.0 | 0 | 0 | 2890 |
| 4.29 | 7a | 40 | 8.0 | 0.0 | 140.0 | 18.8 | 2600 | 0 | 0 |

*WP = weight parts

Extrudates

Pharmaceutical preparations of CGRP antagonist according to the present invention in form of extrudates are also possible. After cutting/spheronizing the extrudates are filled directly into capsules or are used for tablets after grinding.

The method of preparation has following steps:

1. Extrusion

2a. cutting/spheronizing

2b. grinding and subsequently pressing to tablets

### EXAMPLE 5a

Method of preparation of moist extrudates
Composition:

| | |
|---|---|
| Povidone K25 | 6 weight parts |
| Microcrystalline cellulose | 40 weight parts |
| CGRP-antagonist | 100 weight parts |

119 weight parts CGRP-antagonist, 40 weight parts microcrystalline cellulose (Avicel PH 101) and 6 weight parts povidone (Kollidon K25) are mixed for 15 minutes in an adequate mixer. Afterwards the composition is extruded through a twin screw extruder at a rate of 1 kg/h by metered addition of water. The moment of torsion of 19% is controlled by the proportioning of the water. The diameter of the holes of the die base at the end of the extruder 0.8 mm.

The spheronizing of the product is made by a spheronizer, for 3 minutes at approx. 850 RPM.

Drying of the pellets at 80° C. for 1.5 h in a fluid bed dryer.

The material is screened by a tumble screener with different sieves (0.71-1.25 mm). The adequate fractions between 0.71 and 0.90 resp. 0.90 and 1.12 mm are used.

### EXAMPLE 5b

Method of preparation of moist extrudates
Composition:

| | |
|---|---|
| Povidone K25 | 4 weight parts |
| Microcrystalline cellulose | 30 weight parts |
| CGRP-antagonist | 10.0 weight parts |

10.0 weight parts CGRP-antagonist, 30 weight parts microcrystalline cellulose (Avicel PH 101) and 4 weight parts povidone (Kollidon K25) are mixed for 15 minutes in an adequate mixer. Afterwards the composition is extruded through a twin screw extruder at a rate of 1 kg/h by metered addition of water. The moment of torsion of 19% is controlled by the proportioning of the water. The diameter of the holes of the die base at the end of the extruder is 0.8 mm.

The spheronizing of the product is made by a spheronizer, for 3 minutes at approx. 850 RPM.

Drying of the pellets at 80° C. for 1.5 h in a fluid bed dryer.

The material is screened by a tumble screener with different sieves (0.71-1.25 mm). The adequate fractions between 0.71 and 0.90 resp. 0.90 and 1.12 mm are used.

### EXAMPLE 5c

Method of preparation of moist extrudates
Composition:

| | |
|---|---|
| Povidone K25 | 15 weight parts |
| microcrystalline cellulose | 110 weight parts |
| CGRP-antagonist | 400 weight parts |

400 weight parts CGRP-antagonist, 110 weight parts microcrystalline cellulose (Avicel PH 101) and 15 weight parts povidone (Kollidon K25) are mixed for 15 minutes in an adequate mixer. Afterwards the composition is extruded through a twin screw extruder at a rate of 1 kg/h by metered addition of water. The moment of torsion of 19% is controlled by the proportioning of the water. The diameter of the holes of the die base is 0.8 mm.

The spheronizing of the product is made by a spheronizer, for 3 minutes at approx. 850 RPM.

Drying of the pellets at 80° C. for 1.5 h in a fluid bed dryer.

The material is screened by a tumble screener with different sieve die bases (0.71-1.25 mm). The adequate fractions between 0.71 and 0.90 resp. 0.90 and 1.12 mm are used.

The following table shows different compositions of the above described method. In the examples 10-600 weight parts CGRP-antagonist as active form, as a physiologically acceptable salt thereof or a hydrate of said salt is used.

Table of Example 5a-c

| | CGRP- | | *WP | *WP | Polyethylen- |
|---|---|---|---|---|---|
| Ex. | Antagonist No. | *WP | Povidone | Poloxamer | glycol 4000 |
| 5.1 | 6 | 80 | 28.0 | 2.7 | 84 |
| 5.2 | 1a | 110 | 38.5 | 3.7 | |
| 5.3 | 14a | 170 | 59.5 | 5.7 | |
| 5.4 | 6a | 100 | 35.0 | 3.4 | |
| 5.5 | 5 | 80 | 28.0 | 2.7 | |
| 5.6 | 6 | 20 | 7.0 | 0.7 | 21 |
| 5.7 | 4a | 200 | 70.0 | 6.8 | 210 |
| 5.8 | 2a | 40 | 14.0 | 1.4 | 42 |
| 5.9 | 17 | 50 | 17.5 | 1.7 | 52.5 |
| 5.10 | 7 | 70 | 24.5 | 2.4 | 73.5 |
| 5.11 | 12 | 110 | 38.5 | 3.7 | |
| 5.12 | 2 | 600 | 210.0 | 20.3 | |
| 5.13 | 21 | 130 | 45.5 | 4.4 | |
| 5.14 | 5a | 40 | 14.0 | 1.4 | 42 |
| 5.15 | 1 | 160 | 56.0 | 5.4 | |
| 5.16 | 3 | 60 | 21.0 | 2.0 | 63 |
| 5.17 | 14a | 200 | 70.0 | 6.8 | |
| 5.18 | 6 | 80 | 28.0 | 2.7 | 84 |
| 5.19 | 16 | 150 | 52.5 | 5.1 | |
| 5.20 | 3 | 10 | 3.5 | 0.3 | 10.5 |

*WP = Weight parts

### EXAMPLE 6a

Method of preparation of melting extrudates
Composition:

| | |
|---|---|
| Povidone K25 | 6 weight parts |
| Poloxamer | 40 weight parts |
| CGRP-Antagonist | 119 weight parts |

100 Weight parts CGRP_Antagonist, 40 weight parts poloxamer and 6 weight parts povidone are mixed for 15 minutes in an adequate mixer. Afterwards the composition is extruded through a twin screw extruder at a rate of 1 kg/h. The moment of torsion of 19% is controlled by temperature. The diameter of the holes of the die base is 0.8 mm.

The discharging extrudate are cutted and spheronized with an adequate spheronizer for 3 minutes at 40°.

Drying of the pellets at 80° C. for approx. 1.5 h in a fluid bed dryer.

The material is screened by a tumble screener with different sieve die bases (0.71-1.25 mm). The adequate fractions between 0.71 and 0.90 resp. 0.90 and 1.12 mm are used.

### EXAMPLE 6b

Method of preparation of melting extrudates
Composition:

| | |
|---|---|
| Povidone K25 | 2 weight parts |
| Poloxamer | 30 weight parts |
| CGRP-antagonist | 10 weight parts |

10 Weight parts CGRP-antagonist, 30 weight parts poloxamer and 2 weight parts povidone are mixed for 15 minutes in an adequate mixer. Afterwards the composition is extruded through a twin screw extruder at a rate of 1 kg/h. The moment of torsion of 19% is controlled by temperature. The diameter of the holes of the die base is 0.8 mm. The discharging extrudate are cutted and spheronized with an adequate spheronizer for 3 minutes at 40°.

Drying of the pellets at 80° C. for approx. 1.5 h in a fluid bed dryer.

The material is screened by a tumble screener with different sieve die bases (0.71-1.25 mm). The adequate fractions between 0.71 and 0.90 resp. 0.90 and 1.12 mm are used.

### EXAMPLE 6c

Method of preparation of melting extrudates
Composition:

| | |
|---|---|
| Povidone K25 | 18 weight parts |
| Poloxamer | 132 weight parts |
| CGRP-antagonist | 400 weight parts |

400 Weight parts CGRP-antagonist, 132 weight parts poloxamer and 18 weight parts povidone are mixed for 15 minutes in an adequate mixer. Afterwards the composition is extruded through a twin screw extruder at a rate of 1 kg/h. The moment of torsion of 19% is controlled by temperature. The diameter of the holes of the die base is 0.8 mm. The discharging extrudate are cutted and spheronized with an adequate spheronizer for 3 minutes at 40° C.

Drying of the pellets at 80° C. for approx. 1.5 h in a fluid bed dryer.

The material is screened by a tumble screener with different sieve die bases (0.71-1.25 mm). The adequate fractions between 0.71 and 0.90 resp. 0.90 and 1.12 mm are used.

The following table shows different compositions of the above described method. In the examples 10-600 weight parts CGRP-antagonist as active form, as a physiologically acceptable salt thereof or a hydrate of said salt is used.

Table of Example 6a-c

| | CGRP-Antagonist | | *WP | *WP | Polyethylen- |
|---|---|---|---|---|---|
| Ex. | No. | *WP | Povidone | Poloxamer | glycol 4000 |
| 6.1 | 17a | 120 | 6.0 | 31.5 | |
| 6.2 | 7 | 130 | 6.5 | 34.1 | |
| 6.3 | 2a | 90 | 4.5 | 23.6 | 70.88 |
| 6.4 | 3a | 40 | 2.0 | 10.5 | 31.50 |
| 6.5 | 16a | 30 | 1.5 | 7.9 | 23.63 |
| 6.6 | 2 | 20 | 1.0 | 5.3 | 15.75 |
| 6.7 | 16 | 110 | 5.5 | 28.9 | |
| 6.8 | 5a | 180 | 9.0 | 47.3 | |
| 6.9 | 21a | 150 | 7.5 | 39.4 | |
| 6.1 | 3 | 90 | 4.5 | 23.6 | |
| 6.11 | 16 | 190 | 9.5 | 49.9 | |
| 6.12 | 13 | 600 | 30.0 | 157.5 | |
| 6.13 | 5 | 130 | 6.5 | 34.1 | |
| 6.14 | 15 | 150 | 7.5 | 39.4 | |
| 6.15 | 1 | 130 | 6.5 | 34.1 | |
| 6.16 | 4a | 110 | 5.5 | 28.9 | 86.63 |
| 6.17 | 4 | 180 | 9.0 | 47.3 | |
| 6.18 | 5 | 90 | 4.5 | 23.6 | |
| 6.19 | 17 | 150 | 7.5 | 39.4 | |
| 6.20 | 4 | 100 | 5.0 | 26.3 | |
| 6.21 | 1a | 70 | 3.5 | 18.4 | 55.13 |
| 6.22 | 13 | 20 | 1.0 | 5.3 | 15.75 |
| 6.23 | 4 | 200 | 10.0 | 52.5 | |
| 6.24 | 13 | 10 | 0.5 | 2.6 | 7.88 |
| 6.25 | 2 | 30 | 1.5 | 7.9 | 23.63 |

*WP = Weight parts

### EXAMPLE 7

Subsequent Treatment: Production of Tablets

The extrudates are grinded in an adequate mill. The product are used fort the production of tablets (see Example 1 and 2).

Powder Inhalant

Preparation of Spherically Nanostructured Microparticles of the Active Substances for Manufacture of a Powder Inhalant

For the preparation of a solution of 4% by weight the active substance is solved in an ethanol/water (4:1)-mixture and the solution is sprayed in a way resulting a spray mist with a droplet size of the characteristic value ×50 (median value=particle size/droplet size, below which 50% of the quantity of particles are found, with regard to the volume distribution of the individual particles/droplets) in the range between 1.5 and Q_{(5.8)} (corresponding to the quantity of particles below 5.8 μm, based on the distribution by volume of the particles) between 30% and 100%. The resulting spray mist is dried using a drying gas with a inlet temperature of 100° C. to 200° C. and an outlet temperature of 40° C. to 120° C. The volumetric flow of the spray gas of is 1 Nm³/h to 15 Nm³/h and a volumetric flow of the drying gas of 15 Nm³/h to 150 Nm³/h is used. The solid fraction remaining after the solvent has evaporated is separated off from the gas current by means of an inertia force separator (e.g. cyclone) and/or by a filter unit and collected.

### EXAMPLE 8

Capsules for powder inhalation with 0.5 mg CGRP-antagonist
Composition:
1 capsule for powder inhalation contains:

| | |
|---|---|
| CGRP-antagonist | 0.5 mg |
| Lactose | 20 mg |
| Hard gelatine capsule | 50 mg |

Method of Preparation:

The active ingredient in form of spherical nanostructured mircroparticle is mixed homogeneously with lactose. The mixture is subsequently filled into hard gelatine capsules.

This method of preparation is the basic principle of further examples being shown in the following table.

Table of Example 8

| Example | CGRP-Antagonist (n) | mg | mg Lactose |
|---|---|---|---|
| 8.1 | 4 | 30.00 | 80.00 |
| 8.2 | 12 | 10.00 | 60.00 |
| 8.3 | 21 | 20.00 | 70.00 |
| 8.4 | 6 | 30.00 | 80.00 |
| 8.5 | 16 | 25.00 | 75.00 |
| 8.6 | 1 | 30.00 | 80.00 |
| 8.7 | 3 | 20.00 | 70.00 |
| 8.8 | 21 | 10.00 | 60.00 |
| 8.9 | 3 | 20.00 | 70.00 |
| 8.10 | 11 | 0.30 | 50.30 |
| 8.11 | 5 | 0.10 | 50.10 |
| 8.12 | 5 | 30.00 | 80.00 |
| 8.13 | 16 | 30.00 | 80.00 |
| 8.14 | 2 | 3.00 | 53.00 |
| 8.15 | 22 | 20.00 | 70.00 |
| 8.16 | 5 | 5.00 | 55.00 |
| 8.17 | 6 | 20.00 | 70.00 |
| 8.18 | 2 | 10.00 | 60.00 |
| 8.19 | 14 | 10.00 | 60.00 |
| 8.20 | 4 | 0.00 | 50.00 |
| 8.21 | 6 | 10.00 | 60.00 |
| 8.22 | 3 | 15.00 | 65.00 |
| 8.23 | 14 | 10.00 | 60.00 |
| 8.24 | 4 | 50.00 | 100.00 |
| 8.25 | 6 | 30.00 | 80.00 |
| 8.26 | 5 | 0.00 | 50.00 |
| 8.27 | 16 | 20.00 | 70.00 |

### EXAMPLE 9

Injectable Solution with 0.5 mg CGRP-Antagonist

Composition:

CGRP-antagonist 0.5 mg

physiological solution of NaCl

The active ingredient is solved in a physiological solution of NaCl.

The dose is variable, different doses are displayed in the following table. The examples contain 0.2-30 mg of CGRP-antagonist as active form, in form of a physiologically acceptable salt thereof or a hydrate of said salt.

Table of Example 9

| Example | CGRP-Antagonist Nr. | mg |
|---|---|---|
| 9.1 | 5 | 0.20 |
| 9.2 | 4a | 14.30 |
| 9.3 | 16 | 4.40 |
| 9.4 | 6a | 10.30 |
| 9.5 | 6 | 1.80 |
| 9.6 | 3 | 1.30 |
| 9.7 | 2a | 4.40 |
| 9.8 | 12 | 9.40 |
| 9.9 | 4 | 2.60 |
| 9.10 | 12 | 8.20 |
| 9.11 | 21 | 4.30 |
| 9.12 | 5a | 25.50 |
| 9.13 | 6 | 14.20 |
| 9.14 | 11 | 13.40 |
| 9.15 | 1a | 5.40 |
| 9.16 | 15 | 6.90 |

### EXAMPLE 10

Suppositories with 200 mg CGRP-antagonist
Composition:

| | |
|---|---|
| CGRP-antagonist | 238 mg |
| Hard fat | ad 2 g |

Method of Preparation:

The active substance is previously ground and sieved through a suitable sieve and hard fat is added. When prepared by moulding, the medicated mass, sufficiently liquified by heating, is poured into suitable moulds. The suppository solidifies on cooling.

The dose is variable, therefore different doses are displayed in the following table. The examples contain 50-600 mg of CGRP-antagonist as active form, in form of a physiologically acceptable salt thereof or a hydrate of said salt.

Table of Example 10

| Example | CGRP-Antagonist Nr. | mg |
|---|---|---|
| 1.1 | 13 | 250 |
| 1.2 | 6a | 150 |
| 1.3 | 1a | 460 |
| 1.4 | 12 | 540 |
| 1.5 | 6 | 320 |
| 1.6 | 3 | 180 |
| 1.7 | 17 | 150 |
| 1.8 | 3a | 480 |
| 1.9 | 4 | 600 |
| 1.10 | 5 | 180 |

What is claimed is:

1. A method for the treatment of hot flushes in a human male who has undergone castration which method comprises administering, to a male host in need of such treatment, a therapeutically effective amount of a compound selected from the group consisting of
(a) (S)-2-(4-amino-3-chloro-5-trifluoromethyl-benzyl)-1-[4-(4-methyl-piperazin-1-yl)-piperidin-1-yl]-4-[4-(2-oxo-1,2,4,5-tetrahydro-1,3-benzodiazepin-3-yl)-piperidin-1-yl]-butane-1,4-dione,
(b) 4-(2-oxo-1,2,4,5-tetrahydro-1,3-benzodiazepin-3-yl)-piperidine-1-carboxylic acid {(R)-1-(3,4-diethyl-benzyl)-2-[4-(1-methyl-piperidin-4-yl)-piperazin-1-yl]-2-oxo-ethyl}-amide,
(c) 4-(2-oxo-1,2,4,5-tetrahydro-1,3-benzodiazepin-3-yl)-piperidine-1-carboxylic acid (R)-1-(4-amino-3-chloro-5-trifluoromethyl-benzyl)-2-[4-(4-methyl-piperazin-1-yl)-piperidin-1-yl]-2-oxo-ethyl ester,
(d) 4-(2-oxo-1,2,4,5-tetrahydro-1,3-benzodiazepin-3-yl)-piperidine-1-carboxylic acid {(R)-1-(4-amino-3-chloro-5-trifluoromethyl-benzyl)-2-[4-(4-methyl-piperazin-1-yl)-piperidin-1-yl]-2-oxo-ethyl}-amide,
(e) ((S)-2-(4-amino-3,5-bis-trifluoromethyl-benzyl)-1-[4-(4-methyl-piperazin-1-yl)-piperidin-1-yl]-4-[4-(2-oxo-1,2,4,5-tetrahydro-1,3-benzodiazepin-3-yl)-piperidin-1-yl]-butane-1,4-dione,
(f) 4-(2-oxo-1,2,4,5-tetrahydro-1,3-benzodiazepin-3-yl)-piperidine-1-carboxylic acid {(R)-1-(4-amino-3,5-bis-trifluoromethyl-benzyl)-2-[4-(4-methyl-piperazin-1-yl)-piperidin-1-yl]-2-oxo-ethyl}-amide, and
(g) 4-(2-oxo-1,2,4,5-tetrahydro-1,3-benzodiazepin-3-yl)-piperidine-1-carboxylic acid (R)-1-(4-amino-3,5-bis-trifluoromethyl-benzyl)-2-[4-(4-methyl-piperazin-1-yl)-piperidin-1-yl]-2-oxo-ethyl ester,
or a physiologically acceptable salt.
